(19) United States
(12) Patent Application Publication
(10) Pub. No.: US 2009/0264316 A1
(43) Pub. Date: Oct. 22, 2009

(54) Ultraviolet/Ozone Patterned Organosilane Surfaces

(75) Inventors: Eric R. BLOUGH, Huntington, WV (US); Hideyo TAKATSUKI, Huntington, WV (US); Kevin Matthew RICE, Crown City, OH (US); Brian Scott DAY, Salt Rock, WV (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Marshall University Research Corporation, Huntington, WV (US)

(21) Appl. No.: 12/426,078

(22) Filed: Apr. 17, 2009

Related U.S. Application Data
(60) Provisional application No. 61/124,560, filed on Apr. 17, 2008.

(51) Int. Cl. C40B 40/04; C23F 1/00; G01N 33/48; C40B 50/14

(52) U.S. Cl. 506/15; 216/94; 216/39; 422/68.1; 506/30

(57) ABSTRACT

UV/ozone treatment can be used to alter the hydrophobicity of organosilane coated surfaces. Methods are contemplated for producing micropatterned surfaces by coating a surface with an organosilane to produce an organosilane surface; and exposing the organosilane surface to ultraviolet light in the presence of oxygen, wherein the micropatterned organosilane surface is produced without the use of photoresist. Methods for producing substrate-micropatterned surfaces further are also contemplated. Suitable substrates include nucleotides, proteins, carbohydrates, and cells. The organosilane coated devices of the present invention may be used in, for example, arrays.

### CROSS REFERENCE TO RELATED APPLICATION

This application claims the benefit of the filing date of U.S. Provisional Patent Application No. 61/124,560, filed Apr. 17, 2008, which is incorporated herein by reference in its entirety.

### STATEMENT REGARDING FEDERALLY-SPONSORED RESEARCH AND DEVELOPMENT

Statement under MPEP 310. The U.S. Government has a paid-up license in this invention and the right in limited circumstances to require the patent owner to license others on reasonable terms as provided for by the terms of Grant No. EPS 0554328 awarded by the National Science Foundation.

### BACKGROUND OF THE INVENTION

1. Field of the Invention

The present invention relates to methods for producing micropatterned and substrate-micropatterned organosilane surfaces. The present invention further relates to devices containing micropatterned or substrate-micropatterned organosilane surfaces.

The present invention also relates to methods for using devices containing micropatterned or substrate-micropatterned organosilane surfaces.

2. Background Art

The application of biomolecular motors for nanotechnological applications has been hampered by limitations in the ability to accurately control the motion of the moving filaments as they travel across a surface. Given this restraint, several approaches have been developed and utilized over the past few years to create well defined patterns of motor proteins across a micropatterned surface. For example, motor protein binding tracks have been produced by aligned poly(tetrafluoroethylene) films on glass, micrometer scale grooves, and nanoimprinting lithography.

Nanoprinting UV lithography requires the application of photoresist by spin coating, heating to evaporate the excess coating solvent and harden the photoresist, aligning a photomask to the surface, exposing the photoresist, surface and photomask to UV light, applying chemicals to develop the image, heating to stabilize and harden the photoresist, and removing the photoresist with chemicals (Sundberg, M., et al., Langmuir 22:7302-7312 (2006); Sundberg, M., et al. Langmuir 22:7286-7295 (2006)).

The present methods eliminate the need for the use of photoresist. The micropatterned and substrate-micropatterned organosilane surfaces of the present invention can be produced more cheaply, with fewer noxious chemicals, and are less time consuming than previously used methods utilizing UV lithography.

### BRIEF SUMMARY OF THE INVENTION

The present invention relates to methods for producing micropatterned and substrate-micropatterned organosilane surfaces. The present invention also relates to devices containing micropatterned or substrate-micropatterned organosilane surfaces.

This technique is well suited for patterning substrates, including but not limited to proteins and enzymes, onto surfaces. The methods have several advantages: (1) UV/ozone treatment does not require expensive equipment and operates in parallel fashion, (2) the methods can be applied to various materials including silicon, gold and polymer, (3) the methods can be used to pattern surfaces without spotting or using a dispensing machine and has flexibility in pattern shape and size, (4) the efficiency of substrate binding can be altered by adjusting the UV/ozone treatment conditions, (5) the methods can be interfaced with a robotic spotting machine, piezoelectric, electrospray or inkjet dispenser system to immobilize many different substrates on defined patterns, and (6) the methods are simple, reliable and is desirable for mass production.

Ultraviolet (UV) light in the presence of atmospheric oxygen is readily converted to ozone. Ozone is used to produce spatially localized patterning of substrates such as biological motors on an organosilane coated substrate. UV/ozone treatment can be used to alter the hydrophobicity of the organosilane surface, modify substrate binding to the organosilane treated region, and effectively produce localized substrate activity.

### BRIEF DESCRIPTION OF THE DRAWINGS/FIGURES

FIG. 1. Panel A: Schematic depicting mechanism of trimethylchlorosilane (TMCS) binding to glass. Panel B: Surface modification following UV/ozone treatment.

FIG. 2. Figure depicts the water contact angle on UV/ozone treated TMCS glass as a function of time. Lower pictures are representative snapshots of the water droplet on the UV/ozone treated surface.

FIG. 3. The relationship between fluorescence intensity and UV/ozone treatment time on BSA-FITC binding to a TMCS surface. Lower picture are representative images of the fluorescence observed on the UV/ozone treated surface.

FIG. 4. Fluorescent image of protein patterning displayed through BSA-FITC absorption on TMCS coated cover glass following UV/ozone patterning. Upper and lower histograms are cross-sectional profiles at the indicated lines.

FIG. 5. Substrate binding of active heavy meromyosin (HMM) on various surfaces (glass, nitrocellulose, TMCS, and TMCS+UV) as estimated by K/EDTA ATPase assay. * Denotes statistical significance between the compared conditions (P<0.05).

FIG. 6. Velocity of F-actin (μm/sec) over nitrocellulose and TMCS coated surfaces at various HMM (60, 90, 120 μg/ml) concentrations. Scale bar denotes 5 μm.

FIG. 7. UV/ozone treatment of the TMCS coated surface can be used to restrict F-actin motility. Image series of F-actin movement. Final panel represents a composite image of F-actin movement over an 18 sec. observational period. Scale bar denotes 20 μm.

### DETAILED DESCRIPTION OF THE INVENTION

The present invention relates to methods for producing a micropatterned organosilane surface comprising a) coating a surface with an organosilane to produce an organosilane surface; and b) exposing the organosilane surface to ultraviolet light in the presence of oxygen, wherein the micropatterned organosilane surface is produced without the use of photoresist.

The present invention also relates to methods for producing a micropatterned organosilane surface consisting of a) coating a surface with an organosilane to produce an organosilane surface; and b) exposing the organosilane surface to ultraviolet light in the presence of oxygen.

The present invention also relates to methods for producing a substrate-micropatterned organosilane surface consisting of a) coating a surface with an organosilane to produce an organosilane surface; b) exposing the organosilane surface to an ultraviolet light in the presence of oxygen; and c) exposing the micropatterned organosilane surface to a substrate.

The present invention also relates to methods for producing a substrate-micropatterned organosilane surface comprising a) coating a surface with an organosilane to produce an organosilane surface; b) exposing the organosilane surface to ultraviolet light in the presence of oxygen; and c) exposing the micropatterned organosilane surface to a substrate, wherein the substrate micropatterned organosilane surface is produced without the use of photoresist.

### DEFINITIONS

The term “device” as used herein means any material that is capable of being coated with an organosilane. Devices may be made out of material including, but not limited to, glass, silicon, gold, polymers, ceramic, quartz, and combinations thereof.

Examples of devices include coated coverslips, plates, chips, beads, and wafers.

The term “grid” as used herein, means any patterned material that cannot be penetrated by ultraviolet light. An example of a grid is a two-dimensional structure made up of a series of intersecting vertical and horizontal axes. A grid pattern may contain regularly spaced horizontal and vertical lines. The terms “grating,” “grid,” and “photomask” are used interchangeably throughout the specification. Placing a grid on top of an organosilane surface and exposing it to UV/ozone treatment results in a “micropatterned surface.”

The term “organosilane” as used herein, means any organic derivative of a silane containing at least one carbon to silicon bond.

The term “organosilane surface” as used herein, means any surface coated with an organosilane.

The term “photoresist” as used herein, means a light sensitive material used in photolithography. Examples of photoresist include positive photoresist and negative photoresist. Specific examples of photoresist include polymethacrylate (PMMA) and polymethylglutarimide (PMGI).

The term “stable” as used herein, means any micropatterned organosilane surface on which substrate will adhere to after a period of about 14 days, about 21 days, about 28 days, about 14-21 days, about 21-28 days, about 3 months, about 6 months, about 9 months, about 12 months, about 1-3 months, about 3-6 months, about 6-9 months, or about 9-12 months, after UV/ozone treatment.

The term “substrate-micropatterned surface” as used herein, means a coated surface made by exposing an organosilane surface to UV/ozone treatment and then contacting the organosilane coated surface with a substrate.

The term “substrate” as used herein, means any hydrophobic substance that can bind to the micropatterned organosilane surface. Examples of substrates include, but are not limited to, nucleic acids, proteins, carbohydrates, cells, and combinations thereof.

The term “UV/ozone treatment” as used herein, means exposing a surface to ultraviolet light in the presence of oxygen.

### Methods of Producing an Organosilane Coated Surface

Organosilanes contemplated for use in the present invention include trimethylchlorosilane, epoxysilane, dimethoxysilane, triethylsilane, methyltrimethoxysilane, ethyltrimethoxysilane, phenyltrimethoxysilane, dimethyldimethoxysilane, phenylmethyldimethoxysilane, diphenyldimethoxysilane, methyltriethoxysilane, ethyltriethoxysilane, phenyltriethoxysilane, dimethyldiethoxysilane, phenylmethyldiethoxysilane, diphenyldiethoxysilane, orthomethyl silicate, orthoethyl silicate, and combinations thereof. A preferred organosilane is trimethylchlorosilane (TMCS).

Organosilanes can be coated onto a surface by either solution deposition or by chemical vapor deposition (CVD). Solution deposition is performed by submerging a surface to be patterned into a volume of organosilane sufficient to cover the surface for a period of about 1 minute, about 2 minutes, about 5 minutes, about 10 minutes, about 15 minutes, about 20 minutes, about 25 minutes, about 30 minutes, about 45 minutes, about 60 minutes, about 1 to 30 minutes, about 1 to 15 minutes, about 1 to 10 minutes, about 1 to 5 minutes, or about 2 to 5 minutes. Chemical vapor deposition is performed by placing a device in a chamber containing an organosilane saturated vapor for a period of about 1 minute, about 2 minutes, about 5 minutes, about 10 minutes, about 15 minutes, about 20 minutes, about 25 minutes, about 30 minutes, about 45 minutes, about 60 minutes, about 1 to 30 minutes, about 1 to 15 minutes, about 1 to 10 minutes, about 1 to 5 minutes, or about 2 to 5 minutes.

Organosilanes can be coated onto the surface of virtually any self-supporting device. The device can be rigid or flexible, and can be made of glass, plastic, ceramics, silicon, gold, polymers, aluminum, metal, or combinations thereof. Most preferably, the device is made of glass. Examples of devices include coverslips, plates, chips, beads, and wafers. Devices may be any size, including microdevices and nanodevices.

Ultraviolet light can be applied via a handheld Tesla coil or other UV light source. The organosilane surface is exposed to ultraviolet light at a distance of about 1 cm from the UV light source. Exposure is usually to 185 nm UV light. The organosilane surfaces can be treated with UV/ozone for a period of about 15 seconds, about 30 seconds, about 1 minute, about 2 minutes, about 5 minutes, about 10 minutes, about 15 minutes, about 1 to 15 minutes, about 1 to 10 minutes, about 1 to 5 minutes, about 1 to 2 minutes, or about 30 seconds to 1 minute. The organosilane surface is exposed to UV light in the presences of atmospheric oxygen, preferably at room temperature.

The device containing an organosilane surface can be exposed to ultraviolet light such that a pattern is created on the organosilane surface. This can be done by, for example, placing a grid, grating, photomask or other means of obstructing the passage of UV light between an organosilane surface and the source of UV light during UV/ozone treatment. Any pattern or design defined by the grid, grating, photomask, or other means of obstructing the passage of UV light may be used to create the micropatterned surface.

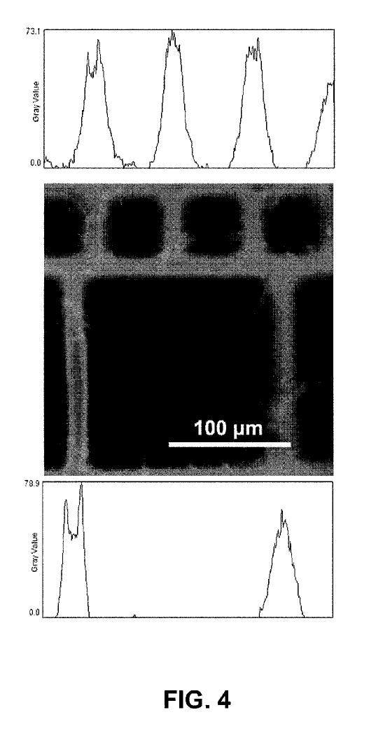

The micropatterned surface may be a pattern that includes grooves or tracks, such as shown in FIG. 4. Ultraviolet light can be applied to create a micropatterned organosilane surface characterized by tracks having a width of about 50 nm to about 50 μm, about 50 nm to about 10 μm, about 50 nm to about 5 μm, about 50 nm to about 1 μm, about 100 nm to about 1 μm, about 200 nm to about 800 nm, about 500 nm to about 1 μm, about 1 μm to about 10 μm, and about 1 μm to about 5 μm.

The methods of the present invention can be performed in the absence of photoresist, such as polymethacrylate (PMMA), polymethylglutarimide (PMGI), or mixtures thereof.

### Substrates

The present invention also includes methods of making a substrate-micropatterned surface. A substrate can be any material that can bind to the micropatterned organosilane surface. Specific examples of substrates for use in the present invention include nucleic acids, proteins, carbohydrates, cells, and combinations thereof. Nucleic acids that may be used in the present invention include DNA, RNA, cDNA, mRNA, miRNA, and DNA oligonucleotides. Proteins that may be used in the present invention include biological motors, such as heavy meromyosin (HMM), myosin, dynein, kinesin, F₁-ATPase, and any other protein that is capable of binding to a hydrophobic surface. Substrates that may also be used in the present invention include carbohydrates, molecules, and/or compounds that are capable of interacting with or binding to a hydrophobic surface.

The term “motor protein” as used herein, means proteins that are capable of moving along a surface of a suitable substrate (or are capable of having another protein move along it) or are capable of generating force or torque in a cell. Examples of motor proteins include myosin, actin, kinesin, tubulin, dynein, dynamin, RNA polymerase, DNA polymerase, helicase, RSC complexes, SMC proteins, topoisomerase, and ATP synthase. Motor proteins are also called biomolecular motors, biological motors, or molecular motors.

Substrates can be adhered to the organosilane surface by contacting the micropatterned organosilane surface with substrate-containing liquid. For example, substrates can be adhered to the micropatterned organosilane substrate by using a flow cell. Flow cells can be created by placing pieces of double sided tape about 7-10 mm apart from each other on a surface and then placing another surface on the top to form a chamber with two open sides. Solutions can be placed in one side of the flow cell and be pulled out of the other side by capillary action using filter paper.

### Uses of Micropatterned Surfaces or Substrate-Micropatterned Surfaces

The surfaces made by the methods of the present invention are useful in any application that requires molecular patterning on a surface, including cell arrays, nucleic acid arrays, protein arrays, and other array types that may be comprised of molecules or compounds that are capable of interacting with or binding to a hydrophobic surface. The methods of the present invention may be interfaced with a robotic spotting machine, piezoelectric, electrospray or inkjet dispenser system to immobilize many different substrates onto a micropatterned organosilane surface.

Other applications include the use of micropatterned surfaces or substrate-micropatterned surfaces for the separation and/or concentration of nucleic acids, proteins, cells, molecules, or compounds that are capable of interacting with or binding to a hydrophobic surface from a liquid, solution or solvent that may be applied or incubated with the patterned surface.

Other applications of note include the use of micropatterned surfaces or substrate-micropatterned surfaces for the development of sensing devices that may use the binding of nucleic acids, proteins, cells, molecules, or compounds that are capable of interacting with or binding to a hydrophobic surface from a liquid or solvent as a means to generate signal input for detection by a sensing system designed to quantitate or measure changes in surface charge, profile, spectral emission, and or ability to absorb or reflect light.

Substrates that may be adhered to the organosilane surfaces include molecular motors. The ability to pattern molecular motors onto a surface increases the use of these molecules for nanotechnological applications. Biological motors can be used to transport micrometer and nanometer-sized objects in the absence of bulk flow. In addition, biological motors use ATP only at the site of action thereby allowing this mode of transport to operate in the absence of external power or bulky pumps.

Another application of the devices of the present invention is their use in in vitro motility assays. In this assay, motors are adhered to a micropatterned surface through their tail domain. The motors then propel microtubules or actin filaments, which can be functionalized with cargo such as biomolecules or nanoparticles, along the surface.

### EXAMPLES

### Materials and Methods

The following materials and methods apply generally to all the examples disclosed herein. Specific materials and methods are disclosed in each example, as necessary.

### Surface Preparation

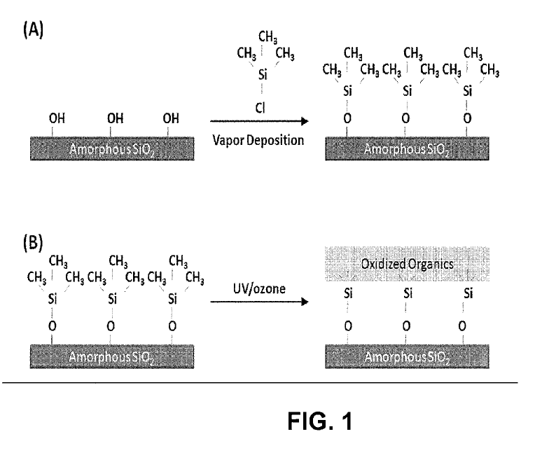

Glass cover slips (Cat. No. 12-540A, Fisher) were functionalized with nitrocellulose (2% collodion in isoamyl acetate, Okenshoji Co, Japan) using solution deposition and soft-baking at 40° C. for 1 hr as detailed previously (Fujita, K. et al., Mol. Cell Biochem. 190:85-90 (1999)). TMCS (trimethylchlorosilane, Sigma Aldrich) was applied using chemical vapor deposition by placing coverslips in a chamber containing a TMCS-saturated atmosphere for 10 min at room temperature (see FIG. 1). In experiments that used UV/ozone treatment, TMCS coated coverslips were exposed to UV light (UVL 20 US-60; 4.5 W; 275 nm; 50 μW/cm², Sen lights Co., Osaka, Japan) in the presence of atmospheric oxygen for different time points as indicated in the text.

### Contact Angle Measurements

The hydrophobicity of TMCS coated cover slips and TMCS-UV/ozone treated cover slips were determined by water contact angle measurements. Contact angles were measured on sessile drops (2 μL) of Nanopure water at room temperature (20-23° C.) in air using a contact angle goniometer constructed from an XY stage fitted with a 20× lens system and a fiber-optic illuminator. The images were captured using a digital camera (Leica DFC280, Germany) and analyzed using the low bond axisymmetric drop shape analysis subroutine in Image J. Observed values were averaged over six different readings.

### Substrate Binding to UV/Ozone-Treated TMCS Surfaces

TMCS coated cover slips were treated with UV/ozone for varying time periods (0, 0.5, 1, 2, 5, or 10 min) and then used in the construction of flow cells using strips of double sided 90 μm thick tape (Nichiban Co, Japan). The resulting chambers had dimensions of approximately 6 mm by 90 μm in cross section. Once assembled, chambers were incubated with BSA-FITC (Bovine Serum Albumin-Fluorescein Isothiocyanate, Molecular Probes, Inc, OR) (5 μg/ml) for 30 min at room temperature (20-23° C.), and then washed three times with motility buffer (25 mM KCl, 2 mM MgCl₂, 0.2 mM CaCl₂, mM Imidazole). The sample was observed with an Olympus fluorescence microscope (Olympus America, USA) fitted with a 100× objective and CCD camera. Images were collected using imaging software (Olympus MicroSuite Basic, Olympus America, USA) using the same exposure period and camera control settings throughout.

### HMM ATPase Activity on UV/Ozone-Treated TMCS Surfaces

Glass coverslips were incubated with heavy meromyosin (HMM) (48 μg/ml) solubilized in K/EDTA ATPase buffer (0.5 M KCl, 5 mM EDTA, 20 mM Tris HCl (pH7.5)) for 10 min on ice and then blocked with 1 mg/ml BSA for 10 min. Once coated, coverslips were incubated with 1.6 ml of K/EDTA ATPase buffer supplemented with 0.5 mM ATP for 30 min at 25° C. Adenosine-5′-triphosate (ATP) hydrolysis by the HMM was determined by measuring the absorbance of aliquots for the presence of inorganic phosphate (Pi) using the malachite green method (Kodama, T., et al. Biochem. 99:1465-1472 (1986)). The number of ATPase-active HMM molecules per unit area were estimated by comparing the ATPase rate to that of a known concentration of HMM in solution.

Protein Patterning with UV/Ozone on TMCS Substrates

To examine the potential of UV/ozone exposure to pattern fine lines, an electron microscope grid (Electron Microscopy Sciences, PA, USA) was placed on a quartz slide (SPI supplies, PA, USA) that was in turn mounted over a TMCS coated coverslip. Once assembled, the cover slip was then placed 1 cm under the UV light for 10 min. Using a flow cell, BSA-FITC (5 μg/ml) was applied to the surface and the solution allowed to incubate for 30 minutes at room temperature (20-23° C.). Excess, unbound BSA-FITC was removed by rinsing the flow cell three times with 50 μl of motility buffer. Protein patterning was observed by epifluorescence using an Olympus fluorescence microscope (Melville, N.Y., USA) fitted with a 40× objective.

### Motility Assays

The uses of the motility assay have been described previously (Fujita, K., et al., Mol. Cell Biochem. 190:85-90 (1999)). Briefly, HMM was extracted from the back and leg muscle of a rabbit and purified as described elsewhere (Samizo, K., et al., Anal. Biochem. 293:212-215 (2001)). HMM was prepared by limited digestion of myosin with α-chymotrypsin (Fujita, K., et al., Mol. Cell Biochem. 190:85-90 (1999)). Actin was extracted in the monomeric form from an actin acetone powder of chicken breast muscle (Kohama, K. J. Biochem. 87:997-999 (1980)) and the polymerized actin filaments were labeled with tetramethylrhodamine-phalloidin (Molecular Probes, Inc., OR, USA). Flow cells were constructed from a No. 2 cover slip using strips of double sided 90 μm thick tape (Nichiban Co, Japan). The flow cell was filled with 60, 90, or 120 μg/ml HMM diluted in the assay buffer containing 25 mM KCl, 2 mM MgCl₂, 0.2 mM CaCl₂ and 25 mM imidazole at pH 7.0 and incubate for 5 min. Bovine serum albumin (0.1% BSA in water) was used to prevent the filaments from nonspecifically binding to the surface. After a 5 min incubation, the flow cell was washed with the assay buffer, and 0.25 μg/ml labeled filaments in assay buffer supplemented with 10 mM dithiothreitol, 4.5 mg/ml D(+)-glucose, 0.22 mg/ml glucose oxidase and 0.036 mg/ml catalase was introduced into the flow cell. Motility was activated by exchange of actin loading solution with assay buffer containing 1.5 mM adenosine-5′-triphosate (ATP). Motility assays were performed at room temperature (20-23° C.) and observed under fluorescence microscope (Olympus BH-2, Japan) with a 100× objective (1.3 N.A., oil-immersion; UVFL100, Olympus, Japan) through a CCD camera (Hamamatsu Photonics C2400, Japan) and a monitor. The images of the filaments were digitally recorded onto a computer (Dell, Dimension 4300). The velocity of actin filament movement was analyzed using software (Image J) as outlined by the manufacturer. Image frames were collected at 0.5 Hz for 30 and used for the determination of sliding velocity.

### Statistical Analysis

Results are presented as mean±SEM. All comparisons were performed by Students t-tests or a two-way analysis of variance (ANOVA) with post hoc analysis where appropriate. For all comparisons the alpha level was set a priori at P<0.05.

### Example 1

### Surface Hydrophobicity with UV/Ozone Exposure

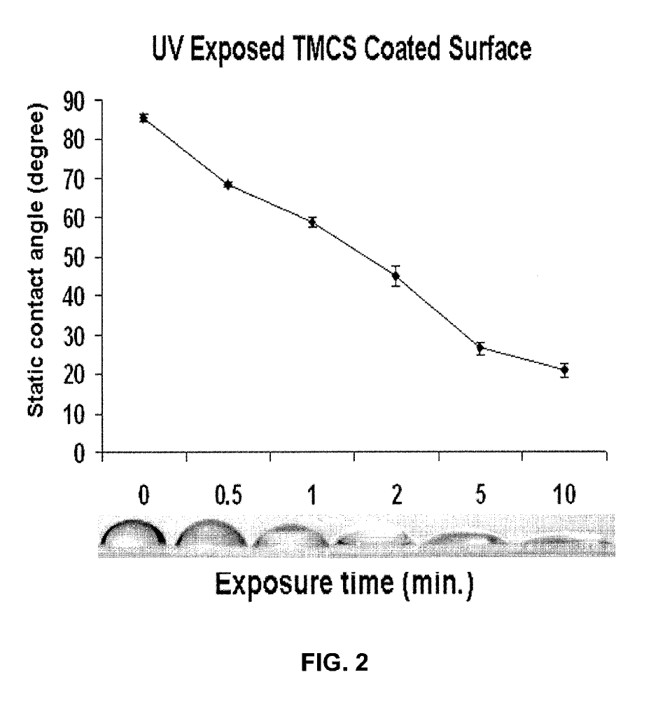

UV/ozone was used to modify the functional groups on the surface of the device. To examine the relationship between UV/ozone treatment and TMCS surface chemistry, the hydrophobicity of treated surfaces was estimated by determining water contact angles. Surfaces were exposed to TMCS by chemical vapor deposition and subjected to UV/ozone treatment (FIG. 1) as described above. With increasing UV/ozone treatment, the water contact angle decreased as expected for the exchange of surface methyl groups for oxidized organic species (FIG. 2). These results indicate that UV/ozone treatment of TMCS surfaces is effective in enhancing surface hydrophilicity.

### Example 2

### Protein Binding to TMCS Following UV Treatment

To determine how UV/ozone treatment affects protein binding to TMCS coated surfaces, FITC labeled BSA was allowed to bind to TMCS coated cover slips that had undergone UV/ozone treatment. Binding affinity was estimated indirectly by digitizing the captured images and then determining the average pixel intensity of twelve randomly positioned regions (2,500 μm²) per flow cell. To allow approximate quantitative comparisons, all imaging parameters (pixel dwell time, optical and electronic filters, and detector gain and offset) were kept constant between samples.

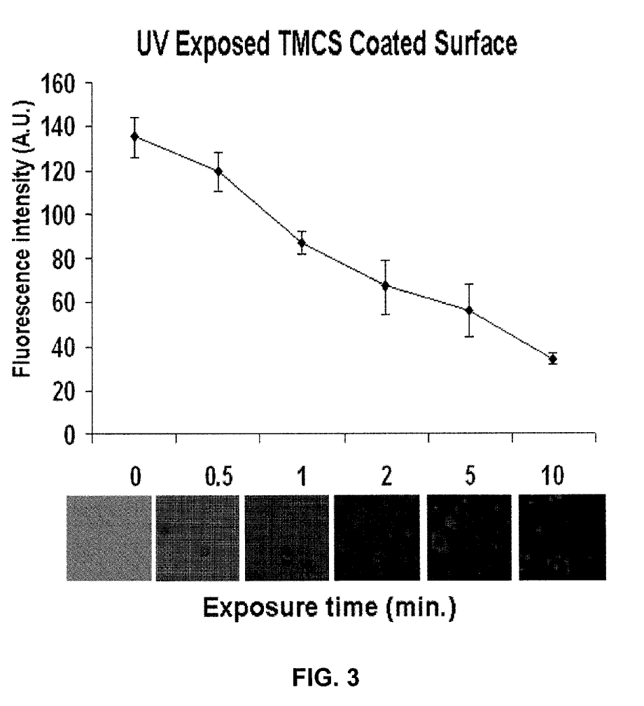

FIG. 3 shows fluorescent intensities of square areas covering the TMCS coated surface. As UV/ozone treatment is increased, the binding of the FITC labeled BSA to the TMCS surface is decreased. Hydrophobic surfaces readily adsorb proteins compared to hydrophilic surfaces. The water contact angle data demonstrates that the surface moves from a hydrophobic to a hydrophilic surface. These data show that UV/ozone treatment can be used to alter the affinity of TMCS for protein.

### Example 3

### Protein Patterning

Control over the patterning of biomolecules on solid surfaces, while preventing nonspecific binding of unwanted areas and species, is an important design consideration for the development of nanotechnological devices. To examine the potential of UV/ozone treatment to produce protein patterning a grating was used to mask the TMCS coated surface. To ensure that any protein patterning that resulted from this procedure was due to the UV/ozone exposure and not due to physical modification or contact, a quartz slide was placed between the grid and the TMCS coated surface. A fluorescent image of the protein patterning acquired with this technique is shown in FIG. 4. These experiments suggest that UV/ozone exposure can be used to effectively prevent non-specific protein binding to a TMCS coated surface.

### Example 4

### Heavy Meromyosin (HMM) Binding to TMCS with UV/Ozone Treatment

To determine how UV/ozone treatment affects myosin II binding to TMCS, myosin ATPase activity was examined directly on untreated and treated TMCS surfaces.

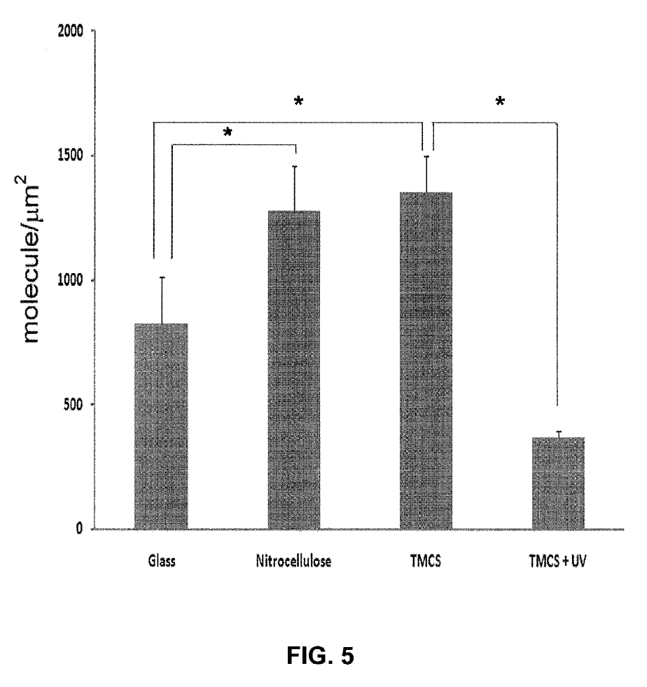

The results show that compared to glass, we observed higher active HMM binding to nitrocellulose and TMCS (glass: 827±188; nitrocellulose: 1,280±179; TMCS 1,354±146 molecules active HMM/μm²; (P<0.05)) (FIG. 5). As expected, UV/ozone treatment produced a significant decrease in active HMM binding to TMCS (TMCS 1,354±146 vs. UV/ozone treated TMCS 372±25 molecules active HMM/μm² (P<0.05)).

### Example 5

### Actin Motility

In our experiments, 3-week old TMCS coated coverslips have similar actomyosin motility to the 2-days old fresh TMCS coverslips (data not shown).

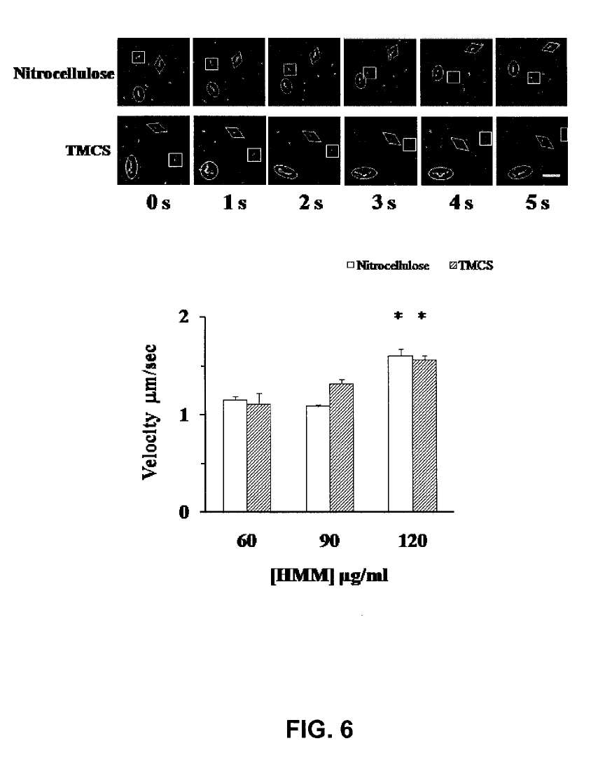

The motility characteristics of actin filaments were examined over several different concentrations of myosin. To ensure the ability to compare across substrates (nitrocellulose, TMCS) all motility assays were performed under identical conditions. Once introduced into the flow cell, the F-actin was found to bind rigidly to the myosin immobilized on the cover slip. On the addition of ATP, the actin moved in a continuous fashion with an average velocity in the range of 1.1-1.6 μm/s at 20-23° C. (FIG. 6). Both nitrocellulose and TMCS appeared to support actin filament motility of each quality and no differences in velocity of movement were noted when comparing across substrates. As expected, the velocity of actin filament movement increased with increasing myosin concentration. Taken together, these data support previous reports examining actin-filament motility on TMCS and confirm that both TMCS and nitrocellulose support actin filament motility of comparable quality.

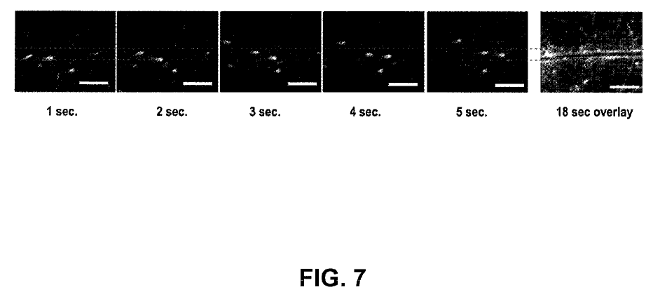

It was determined whether UV/ozone treatment could be used to pattern TMCS coated surfaces and place active myosin II at discrete locations. For these experiments, a grating identical to that used for the protein deposition studies was used to mask the TMCS coated surface prior to UV/ozone treatment for 5 min (FIG. 7). Once treated, the effect of UV/ozone treatment of TMCS surface on actin filament motility was determined using the motility assay. As expected, predominant actin filament motility was observed only on those areas that had not received UV exposure.

Taken together, these data show that UV/ozone treatment can be used to manipulate the amount and position of active biomolecular motors across a micropatterned organosilane surface without the use of photoresist.

What is claimed is:

1. A method of producing a micropatterned organosilane surface comprising a) coating a surface with an organosilane to produce an organosilane surface; and b) exposing the organosilane surface to ultraviolet light in the presence of oxygen, wherein the micropatterned organosilane surface is produced without the use of photoresist.

2. The method of claim 1 wherein the organosilane is selected from the group consisting of trimethylchlorosilane, epoxysilane, dimethoxysilane, triethylsilane, methyltrimethoxysilane, ethyltrimethoxysilane, phenyltrimethoxysilane, dimethyldimethoxysilane, phenylmethyldimethoxysilane, diphenyldimethoxysilane, methyltriethoxysilane, ethyltriethoxysilane, phenyltriethoxysilane, dimethyldiethoxysilane, phenylmethyldiethoxysilane, diphenyldiethoxysilane, orthomethyl silicate, orthoethyl silicate, and combinations thereof.

3. The method of claim 1 wherein a grid is placed on the organosilane surface prior to exposure to said ultraviolet light wherein upon exposure of the grid and organosilane surface to ultraviolet light, a pattern is produced on the organosilane surface.

4. The method of claim 1 wherein the surface is contained on a coverslip, plate, chip, bead, or wafer.

5. The method of claim 1 further comprising exposing the micropatterned organosilane surface to at least one substrate.

6. The method of claim 5 wherein the substrate is a protein, nucleic acid, carbohydrate, cell, or combinations thereof.

7. A device containing a micropatterned organosilane surface made by the method of claim 1.

8. The device of claim 7 wherein the micropatterned organosilane surface remains stable for about 14-21 days.

9. A method for producing a micropatterned organosilane surface consisting of a) coating a surface with an organosilane to produce an organosilane surface; and b) exposing the organosilane surface to ultraviolet light in the presence of oxygen.

10. The method of claim 9 wherein the organosilane is selected from the group consisting of trimethylchlorosilane, epoxysilane, dimethoxysilane, triethylsilane, methyltrimethoxysilane, ethyltrimethoxysilane, phenyltrimethoxysilane, dimethyldimethoxysilane, phenylmethyldimethoxysilane, diphenyldimethoxysilane, methyltriethoxysilane, ethyltriethoxysilane, phenyltriethoxysilane, dimethyldiethoxysilane, phenylmethyldiethoxysilane, diphenyldiethoxysilane, orthomethyl silicate, orthoethyl silicate, and combinations thereof.

11. The method of claim 9 wherein a grid is placed on the organosilane surface prior to exposure to ultraviolet light wherein upon exposure of the grid and organosilane surface to the ultraviolet light, a pattern is produced on the organosilane surface.

12. The method of claim 9 wherein the surface is contained on a coverslip, plate, chip, bead or wafer.

13. A device containing a micropatterned organosilane surface made by the method of claim 9.

14. The device of claim 13 wherein the micropatterned organosilane surface remains stable for about 14-21 days.

15. A method for producing a substrate-micropatterned organosilane surface consisting of a) coating a surface with an organosilane to produce an organosilane surface; b) exposing the organosilane surface to ultraviolet light in the presence of oxygen; and c) exposing the micropatterned organosilane surface to at least one substrate.

16. The method of claim 15 wherein the organosilane is selected from the group consisting of trimethylchlorosilane, epoxysilane, dimethoxysilane, triethylsilane, methyltrimethoxysilane, ethyltrimethoxysilane, phenyltrimethoxysilane, dimethyldimethoxysilane, phenylmethyldimethoxysilane, diphenyldimethoxysilane, methyltriethoxysilane, ethyltriethoxysilane, phenyltriethoxysilane, dimethyldiethoxysilane, phenylmethyldiethoxysilane, diphenyldiethoxysilane, orthomethyl silicate, orthoethyl silicate, and combinations thereof.

17. The method of claim 15 wherein the surface is contained on a coverslip, plate, chip, bead, or wafer.

18. The method of claim 15 wherein the substrate is a protein, nucleic acid, carbohydrate, or cell.

19. The method of claims 6 or 19 wherein the protein is a motor protein.

20. The method of claims 6 or 19 wherein the protein is selected from the group consisting of heavy meromyosin (HMM), myosin, dynein, kinesin, and F₁-ATPase.

21. A device containing a micropatterned organosilane surface made by the method of claim 15.

22. The device of claim 21 wherein the micropatterned organosilane surface remains stable for about 14-21 days.

23. The device as in claims 7, 13, or 22, wherein the device is suitable for use as an array, to separate molecules, to concentrate molecules, in a sensing device, or for use in an in vitro motility assay.
